(19) United States
(12) Patent Application Publication
(10) Pub. No.: US 2026/0357177 A1
(43) Pub. Date: Nov. 12, 2026

(54) ENZYMATIC HYDROLYSIS OF A CELLULOSE-BASED MATERIAL

(71) Applicant: UPM-KYMMENE CORPORATION, Helsinki (FI)

(72) Inventors: Juha TAMPER, Helsinki (FI); Marina STÅHL, Helsinki (FI); Tuuli-Maaria RÖNKÄ, Helsinki (FI)

(21) Appl. No.: 19/142,244

(22) PCT Filed: Dec. 11, 2023
(86) PCT No.: PCT/FI2023/050675

§ 371 Date: Jun. 23, 2025

(30) Foreign Application Priority Data
Dec. 29, 2022 (FI) 20226173

(51) Int. Cl. C12M 1/00; C12M 1/34; C12M 1/40

(52) U.S. Cl. CPC C12M 23/58; C12M 21/18; C12M 29/00; C12M 41/44

(57) ABSTRACT

A process for conducting enzymatic hydrolysis of a cellulose-based material in at least two parallel reactors is disclosed. The hydrolysed material is removed from at least one reactor and the cellulose-based material is fed into at least one reactor while controlling the filling level of the reactors such that in none of the at last two reactors the filling level is below a value of 40% of the reactor volume. During the process there is a time period for each of the reactors when no cellulose-based material is fed into the reactor or hydrolysed material is removed from the reactor at the same time. Further is disclosed an arrangement.

### FIELD OF THE INVENTION

The present disclosure relates to a process for conducting enzymatic hydrolysis of a cellulose-based material to produce hydrolysed material. Further, the present disclosure relates to an arrangement for conducting enzymatic hydrolysis of a cellulose-based material to produce hydrolysed material.

### BACKGROUND OF THE INVENTION

Enzymatic hydrolysis may be considered as a process in which enzymes facilitate the cleavage of bonds in molecules with the addition of the elements of water. Enzymatic hydrolysis may be conducted as a batch-type process or in continuous manner. A typical batch enzymatic hydrolysis process of cellulose-based material can be run such that firstly batch reactors are filled one by one and after the actual enzymatic hydrolysis each batch reactor is emptied one by one followed by washing the batch reactor before the next hydrolysis round. The total tank volume available is thus not very effectively used and a lot of washing sequences, generating a wastewater stream, are required. There thus remains a need for a more efficient way to conduct enzymatic hydrolysis of cellulose-based material.

### SUMMARY

A process for conducting enzymatic hydrolysis of a cellulose-based material to produce hydrolysed material is disclosed. The process comprises:
providing at least two parallel reactors;
subjecting cellulose-based material to enzymatic hydrolysis in each of the at least two reactors;
removing hydrolysed material from at least one reactor during a time interval A;
feeding cellulose-based material into at least one reactor during a time interval B;
controlling that in none of the at least two reactors the filling level of the reactor is below a value of 40% of the reactor volume; and
wherein during the process there is a time period for each of the reactors when no cellulose-based material is fed into the reactor or hydrolysed material is removed from the reactor at the same time.

Further is disclosed an arrangement for conducting enzymatic hydrolysis of a cellulose-based material to produce hydrolysed material. The arrangement comprises:
at least two parallel reactors, wherein each of the reactors comprises an outlet (3a, 3b . . . ) configured to remove hydrolysed material from the reactor, and an inlet (2a, 2b . . . ) configured to feed cellulose-based material into the reactor; and wherein each of the at least two reactors is configured to subject cellulose-based material to enzymatic hydrolysis;
a measurement system configured to produce measurement information indicative of the filling level in each of the reactors; and
a control system configured to control that in none of the at least two reactors the filling level of the reactor is below a value of 40% of the reactor volume, and to control that each of the reactors has a time period during which no cellulose-based material is fed into the reactor or hydrolysed material is removed from the reactor at the same time.

### BRIEF DESCRIPTION OF THE DRAWINGS

The accompanying drawings, which are included to provide a further understanding of the process and the arrangement and constitute a part of this specification, illustrate embodiments and together with the description help to explain the principles of the above. In the drawings:

FIG. 1a illustrates conducting enzymatic hydrolysis of a cellulose-based material to produce hydrolysed material according to comparative example 1;

FIG. 1b illustrates conducting enzymatic hydrolysis of a cellulose-based material to produce hydrolysed material according to example 1a;

FIG. 1c illustrates conducting enzymatic hydrolysis of a cellulose-based material to produce hydrolysed material according to example 1b;

FIG. 2a illustrates conducting enzymatic hydrolysis of a cellulose-based material to produce hydrolysed material according to comparative example 2;

FIG. 2b illustrates conducting enzymatic hydrolysis of a cellulose-based material to produce hydrolysed material according to example 2a;

FIG. 2c illustrates conducting enzymatic hydrolysis of a cellulose-based material to produce hydrolysed material according to example 2b;

FIG. 3a illustrates conducting enzymatic hydrolysis of a cellulose-based material to produce hydrolysed material according to comparative example 3;

FIG. 3b illustrates conducting enzymatic hydrolysis of a cellulose-based material to produce hydrolysed material according to example 3; and

FIG. 4 illustrates an arrangement according to one embodiment.

### DETAILED DESCRIPTION

A process for conducting enzymatic hydrolysis of a cellulose-based material to produce hydrolysed material is disclosed. The process comprises:
providing at least two parallel reactors;
subjecting cellulose-based material to enzymatic hydrolysis in each of the at least two reactors;
removing hydrolysed material from at least one reactor during a time interval A;
feeding cellulose-based material into at least one reactor during a time interval B;
controlling that in none of the at least two reactors the filling level of the reactor is below a value of 40% of the reactor volume; and
wherein during the process there is a time period for each of the reactors when no cellulose-based material is fed into the reactor or hydrolysed material is removed from the reactor at the same time.

Further is disclosed an arrangement for conducting enzymatic hydrolysis of a cellulose-based material to produce hydrolysed material. The arrangement comprises:
at least two parallel reactors, wherein each of the reactors comprises an outlet (3a, 3b . . . ) configured to remove hydrolysed material from the reactor, and an inlet (2a, 2b . . . ) configured to feed cellulose-based material into the reactor; and wherein each of the at least two reactors is configured to subject cellulose-based material to enzymatic hydrolysis;
a measurement system configured to produce measurement information indicative of the filling level in each of the reactors; and
a control system configured to control that in none of the at least two reactors the filling level of the reactor is below a value of 40% of the reactor volume, and to control that each of the reactors has a time period during which no cellulose-based material is fed into the reactor or hydrolysed material is removed from the reactor at the same time.

Feeding cellulose-based material into at least one reactor and removing hydrolysed material from at least one reactor may be conducted at the same time or at a different time.

By the term “time interval A” should be understood in this specification, unless otherwise stated, as time interval during which hydrolysed material is removed from at least one reactor. By the term “time interval B” should be understood in this specification, unless otherwise stated, as time interval during which cellulose-based material is fed into at least one reactor. The time interval A and time interval B may be equal or different. I.e. the duration of feeding cellulose-based material into a reactor (time interval B) may be as long as the duration of removing hydrolysed material from a reactor (time interval A). Thus, the time interval A may be the same as time interval B or they may be different.

The time period (to which each of the reactors is subjected to), during which no cellulose-based material is fed into the reactor nor any hydrolysed material is removed from the reactor at the same time, may vary depending on the mode or manner that the process is conducted. The time period may be 10 min-40 h, or 30 min-30 h, or 1-20 h, or 2-10 h. The time period may depend on the size of the reactors.

At least two parallel reactors are provided. By the term “at least two parallel reactors” should be understood in this specification, unless otherwise stated, that the at least two reactors are taken to be in use at the same time, but there is no material being transferred from one reactor to another. I.e. hydrolyzed material or partly hydrolyzed material is not transferred directly from one reactor to another.

The term “reactor volume” should be understood in this specification, unless otherwise stated, as the total volume of the reactor.

Enzymatic hydrolysis may be considered as a process in which enzymes facilitate the cleavage of bonds in molecules with the addition of the elements of water. In enzymatic hydrolysis of cellulose-based material, cellulose chains are broken into glucose molecules by enzymes.

The cellulose-based material may originate from biomass. The cellulose-based material may comprise or consist of lignocellulose from different origin.

In one embodiment, the cellulose-based material originates from wood, such as hardwood, softwood, or their combination. I.e. the cellulose-based material may be a cellulose-based wood material. The wood material may e.g. originate from pine, poplar, beech, aspen, spruce, eucalyptus, ash, or birch. The wood-based raw material may also be any combination or mixture of these. The wood-based raw material may be broadleaf wood. Preferably the wood-based raw material is broadleaf wood due to its relatively high inherent sugar content, but the use of other kinds of wood is not excluded. The broadleaf wood may be selected from a group consisting of beech, birch, ash, oak, maple, chestnut, willow, poplar, and any combination of mixture thereof.

In general, wood and wood-based materials are essentially composed of cellulose, hemicellulose, lignin, and extractives. Cellulose is a polysaccharide consisting of a chain of glucose units. Hemicellulose comprises polysaccharides, such as xylan, mannan, arabinan, galactan, and glucan. Cellulose is an insoluble linear polymer of repeating glucose units linked by β-1-4-glucosidic bonds. When cellulose-based material is subjected to enzymatic hydrolysis, cellulose chains are cleaved off by means of cleaving off at least one β-1-4-glucosidic bond. Enzymatic hydrolysis may thus result in the formation of a solid lignin residue and a carbohydrate fraction. The carbohydrate fraction may comprise C6 sugars (C₆H₁₂O₆ or (C₆(H₂O)ₙ). The carbohydrate fraction may comprise monosaccharides (C₆H₁₂O₆ or C₅H₁₀O₅), disaccharides (C₁₂H₂₂O₁₁ or C₁₀H₂₀O₁₀), oligosaccharides, and/or polysaccharides ((C₆H₁₀O₅)ₙ or (C₅H₈O₄)ₙ). In one embodiment, the carbohydrate fraction comprises structural units such as galactose, glucose, mannose, arabinose, xylose, glucuronic acid and/or galacturonic acid.

The cellulose-based material may be provided starting e.g. from a wood-based feedstock originating from wood-based raw material and comprising wood chips, that is subjected to pretreatment. By the expression “pretreating” or “pretreatment” should be understood in this specification, unless otherwise stated, (a) process(es) conducted to convert wood-based feedstock to a fraction of cellulose-based material that may be subjected to enzymatic hydrolysis. Pretreatment of the wood-based feedstock may comprise different pretreatment steps. During the different pretreatment steps the wood-based feedstock as such changes. The pretreatment may comprise one or more of the following: mechanical treatment of wood-based material, pre-steaming of the wood-based feedstock, impregnation treatment of the wood-based feedstock, and the steam explosion treatment of the wood-based feedstock.

The enzymatic hydrolysis may be carried out at a temperature of 30-70 °C, or 35-65 °C, or 40-60 °C, or 45-55 °C, or 48-53 °C. Atmospheric pressure may be used. The pH of the material in each of the reactors may be at a pH value of 3.5-6.5, or 4.0-6.0, or 4.5-5.5. The pH of the material in the reactors can be adjusted with the addition of alkali and/or acid. Enzymes are catalysts for the enzymatic hydrolysis. The enzymatic reaction decreases the pH and by shortening the length of the cellulose fibers it may also decrease the viscosity. Subjecting the cellulose-based material to enzymatic hydrolysis may result in cellulose being transformed into glucose monomers with enzymes. Lignin present in the cellulose-based material may remain essentially in solid form.

At least one enzyme may be used for carrying out the enzymatic hydrolysis. The at least one enzyme may be selected from a group consisting of cellulases, hemicellulases, laccases, and lignolytic peroxidases. Cellulases are multi-protein complexes consisting of synergistic enzymes with different specific activities that can be divided into exo- and endo-cellulases (glucanase) and β-glucosidase (cellobiose). The enzymes may be either commercially available cellulase mixes or onsite manufactured.

The enzymatic hydrolysis may be carried out by subjecting the cellulose-based material to enzymatic hydrolysis in the reactor, after which the hydrolysed material may be separated into a solid fraction, which may comprise lignin and non-hydrolyzed cellulose, and a liquid hydrolysate fraction comprising carbohydrates.

The average hydrolysis time may be considered as the time given for the enzymatic hydrolysis of cellulose-based material to take place in the reactor. The average hydrolysis time may be taken as the time calculated from the moment when cellulose-based material is fed into the reactor until the moment when the hydrolysed material is removed from the reactor. The average hydrolysis time in each of the reactors may be at least 6 hours, or at least 8 hours, or at least 10 hours, or at least 12 hours, or at least 24 hours, but not more than 90 hours, or more than 80 hours, or more than 72 hours, or more than 60 hours, or more than 48 hours, or more than 36 hours. The average hydrolysis time in each of the reactors may be 6-90 hours, or 8-80 hours, or 10-72 hours, or 12-65 hours, or 24-48 hours. The average hydrolysis time in each of the reactors may be 6-60 hours, or 8-48 hours, or 10-36 hours. The predetermined residence time may be 6-90 hours, or 12-80 hours, or 24-72 hours. The average hydrolysis time in each of the reactors may be 50-70 hours, or 53-67 hours, or 55-65 hours, or 58-62 hours.

The inventors surprisingly found out that by the process as disclosed in the current specification, one may provide enzymatic hydrolysis of cellulose-based material for an average hydrolysis time that is longer than used in traditional enzymatic hydrolysis. The inventors also surprisingly found out that with the process as disclosed in the current specification, one is able to conduct the enzymatic hydrolysis without any standby time for washing with emptying and filling of any of the at least two parallel reactors during the method or process. Thus, a more efficient manner to conduct enzymatic hydrolysis of cellulose-based material may be achieved.

The hydrolysed material may be removed in an amount such that the filling level of the reactor remains at a value of at least 40%, or at least 50%, or at least 60%, or at least 70%, or at least 80%, or at least 90%, of the reactor volume.

Thus, the amount of material, comprising cellulose-based material, hydrolysed material, or their combination or mixture, depending on the stage of the process, in each of the reactors is kept such that the filling level of the reactor is at a value of at least 40% of the reactor volume. In this specification, unless otherwise stated, the term “filling level” may thus refer to the height at which the material volume is in the reactor compared to the height of the reactor. Thus, the at least two parallel reactors are not emptied in such an amount of material that the surface of the material would be below said set or predetermined value.

In one embodiment, the filling level of each of the reactors is kept at an essentially constant value throughout the process. This is the situation if removing hydrolysed material from a reactor is carried out in the same reactor into which cellulose-based material is simultaneously being fed.

In one embodiment, the filling level of each of the at least two reactors may vary during the process. This is the situation if hydrolysed material is removed from a reactor, which is different from the reactor into which cellulose-based material is simultaneously being fed.

The process may comprise constantly keeping the filling level in each of the reactors at a value of 40-99%, or 60-95%, or 65-90%, or 70-85%, or 75-80%, of the reactor volume. The process may comprise constantly keeping the filling level in each of the reactors at a value of 85-99%, or 90-98%, or 92-96%, of the reactor volume. In one embodiment, the filling level of each of the at least two reactors is never below a value of 40%, or at least 50%, or at least 60%, or at least 70%, or at least 80%, or at least 90%, of the reactor volume.

The reactors may be batch-type reactors. One may provide 2-20, or 2-15, or 2-10, or 2-9, or 2-6, parallel reactors. Alternatively, one may provide 2-20, or 3-15, or 4-10, or 5-9, or 6-8, parallel reactors.

The average flow rate for feeding cellulose-based material into a reactor and for removing hydrolysed material from a reactor may be essentially equal. The flow rate to be used may be calculated based on the volume of the reactors used. The volume of all of the at least two reactors may be essentially the same or equal. As an examples only, hydrolysed material may be removed from the at least one reactor at a flow rate of 5-500 m³/h, or 10-400 m³/h, or 15-300 m³/h, or 20-250 m³/h. In addition, as an example only, cellulose-based material is fed into the at least one reactor at a flow rate of 5-500 m³/h, or 10-400 m³/h, or 15-300 m³/h, or 20-250 m³/h.

In one embodiment, the cellulose-based material is subjected to enzymatic hydrolysis in each of the at least two reactors for a predetermined time during which no hydrolysed material is removed from said reactor nor is any cellulose-based material fed into said reactor.

Removing hydrolysed material from at least one reactor and feeding cellulose-based material into at least one reactor may be carried out such that there is at least one reactor at a time from which no hydrolysed material is removed from and/or to which no cellulose-based material is fed into. Removing hydrolysed material from at least one reactor and feeding cellulose-based material into at least one reactor may be carried out such that there is at least one reactor at a time from which no hydrolysed material is removed from and to which no cellulose-based material is fed into. Removing hydrolysed material from at least one reactor and feeding cellulose-based material into at least one reactor may be carried out such that there is at least one reactor at a time from which no hydrolysed material is removed from or to which no cellulose-based material is fed into.

The process may comprise providing at least three parallel reactors and removing hydrolysed material simultaneously from more than one reactor during the time interval A. The process may comprise providing at least three parallel reactors and feeding cellulose-based material simultaneously into more than one reactor during the time interval B.

Removing hydrolysed material from a reactor and feeding cellulose-based material into a reactor, may be carried out simultaneously in one and the same reactor.

Hydrolysed material may be removed from at least one reactor while simultaneously cellulose-based material is fed into at least one reactor, which is different from the reactor from which hydrolysed material is being removed.

Removing hydrolysed material from at least one reactor and feeding cellulose-based material into at least one reactor may be carried out such that a preceding reactor is fed with cellulose-based material while hydrolysed material is simultaneously removed from a following reactor in the line of parallel reactors. The reactor(s) from which hydrolysed material is removed from and the reactor(s) into which cellulose-based material is fed into may be consecutive reactors but they do not have to be.

The total consistency in each of the reactors is remained essentially constant throughout the process. The total consistency may be e.g. 5-25 weight-% based on the total dry matter content. Keeping the total consistency essentially constant throughout the process has the added utility of making the process easier to control and observe.

The arrangement comprises a measurement system that is configured to produce measurement information indicative of the filling level in each of the reactors. Thus, the filling level of each of the reactors is controlled and adjusted when needed. Any suitable measurement system may be used to measure the filling level in each of the reactors.

The arrangement also comprises a control system configured to control that in none of the at least two reactors the filling level of the reactor is below a value of 40% of the reactor volume, and to control that each of the reactors has a time period during which no cellulose-based material is fed into the reactor or hydrolysed material is removed from the reactor at the same time. The control system may be a single control system configured to control both that in none of the at least two reactors the filling level of the reactor is below a value of 40% of the reactor volume, and that each of the reactors has a time period during which no cellulose-based material is fed into the reactor or hydrolysed material is removed from the reactor at the same time. Alternatively, the control system may comprise a first control subsystem configured to control that in none of the at least two reactors the filling level of the reactor is below a value of 40% of the reactor volume and a second control subsystem configured to control that each of the reactors has a time period during which no cellulose-based material is fed into the reactor or hydrolysed material is removed from the reactor at the same time.

The control system may thus be configured to adjust the removal of hydrolysed material from each of the at least two parallel reactors and the feeding of cellulose-based material into each of the at least two parallel reactors in order to ensure that in none of the at least two parallel reactors the filling level of the reactor is below a value of 40% of the reactor volume. The adjustment of the filling level may be conducted by opening and/or closing valves such that material is feed into and/or removed from a reactor by using pumps.

The process as disclosed in the current disclosure has the added utility that a more effective enzymatic hydrolysis of cellulose-based material may be achieved. The process as disclosed in the current disclosure has the added utility of one being able to operate with full reactors all the time and without the need to wash the reactors as often as with traditional enzymatic hydrolysis processes. Thus, the process has the added utility of providing e.g. 25-30% longer enzymatic hydrolysis time or a similar number of increase in the production capacity than with traditional process and equal tank volume. The capacity may be increased by increasing the flow rate of feeding cellulose-based material and removing hydrolysed material. Thus, if the average hydrolysis time is to be kept the same, the added utility of the method may be achieved in increased flow rate for increasing the capacity.

The process as disclosed in the current disclosure has the added utility of having a minimized risk of enzyme contamination because the reactors are closed and in use during the whole process. Thus, the possibility of contaminants that might be introduced into the reactors during the washing procedure is eliminated.

### EXAMPLES

Reference will now be made in detail to the described embodiments, examples of which are illustrated in the accompanying drawings.

The description below discloses some embodiments in such a detail that a person skilled in the art is able to utilize the method based on the disclosure. Not all steps of the embodiments are discussed in detail, as many of the steps will be obvious for the person skilled in the art based on this specification.

For reasons of simplicity, item numbers will be maintained in the following exemplary embodiments in the case of repeating components.

FIG. 4 illustrates an arrangement for conducting enzymatic hydrolysis of a cellulose-based material to produce hydrolysed material according to one embodiment. The arrangement as illustrated in FIG. 4 comprises four parallel reactors 1a, 1b, 1c, 1d. Each of the four reactors 1a, 1b, 1c, 1d is configured to subject cellulose-based material to enzymatic hydrolysis. Each of the four reactors 1a, 1b, 1c, 1d comprises an outlet 3a, 3b, 3c, 3d configured to remove hydrolysed material from the reactor, and an inlet 2a, 2b, 2c, 2d configured to feed cellulose-based material into the reactor. Further, the arrangement comprises a measurement system 4 configured to produce measurement information indicative of the filling level in each of the reactors 1a, 1b, 1c, 1d. The arrangement further comprises a control system 5 configured to control that in none of the four reactors 1a, 1b, 1c, 1d the filling level of the reactor is below a value of 40% of the reactor volume, and to control that each of the four reactors 1a, 1b, 1c, 1d has a time period during which no cellulose-based material is fed into the reactor or hydrolysed material is removed from the reactor at the same time. For illustrative purposes only, the measurement system 4 and the control system 5 are presented in FIG. 4 as a box surrounding the four parallel reactors 1a, 1b, 1c, 1d.

### Comparative Example 1—Producing Hydrolysed Material

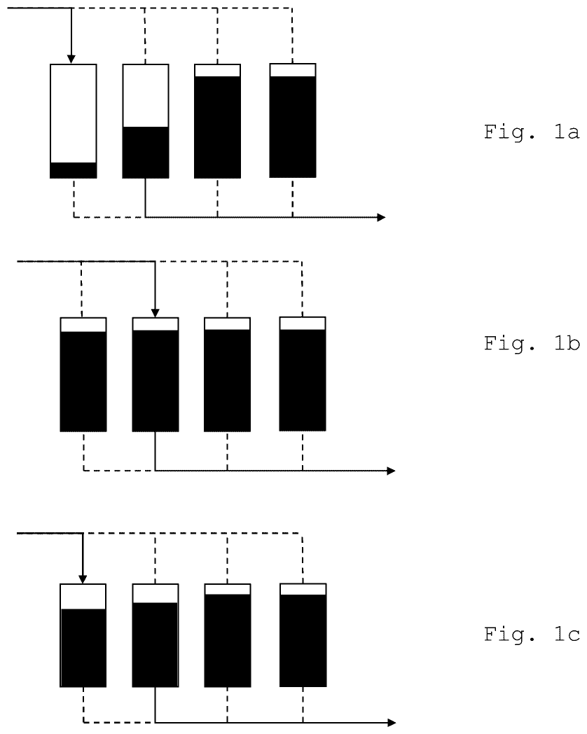

In this comparative example hydrolysed material was produced by enzymatic hydrolysis of cellulose-based material in a manner as disclosed in FIG. 1a.

The enzymatic hydrolysis was in all of the examples conducted with the following conditions and parameters:
Enzyme used: A cellulase mixture that was added to the feed of the cellulose-based material (derived from wood material) and fed together into the reactor.
Hydrolysis temperature and pH: 50 °C, pH 5
Total dry matter content: 14%

The following parameters were used in this comparative example:

TABLE 1

| | |
|---|---|
| Number of parallel reactors | 4 |
| Volume of each of the reactors | 1000 m³ |
| Time interval B | 15 hours |
| (feeding time) | |
| Time interval A | 15 hours |
| (removing time) | |
| Flow rate | 60 m³/h |
| Filling level of the reactor | 90%* |
| volume | |
| Cleaning/standby time | 3 hours |
| Time between feeding and | 27 hours |
| removing | |
| Average hydrolysis time | 42 hours |
| for the whole concept of | |
| four parallel reactors | |

*In those reactors where enzymatic hydrolysis was ongoing, and no hydrolysed material was removed nor cellulose-based material was fed

In this comparative example cellulose-based material was fed into the reactors with continuous feeding and emptying of the reactors one by one. One reactor at a time was emptied, cleaned, and the re-filled. I.e. a reactor was completely emptied from hydrolysed material and after the cleaning of the reactor, it was re-filled or fed with cellulose-based material. The reactors were filled up to a value of 90% of the reactor volume.

### Example 1a—Producing Hydrolysed Material

In this example hydrolysed material was produced by enzymatic hydrolysis of cellulose-based material in a manner as disclosed in FIG. 1b. The following parameters were used:

TABLE 2

| | |
|---|---|
| Number of parallel reactors | 4 |
| Volume of each of the reactors | 1000 m³ |
| Feeding/removing time | 2 hours |
| Flow rate | 60 m³/h |
| Filling level of the reactor | 90% all the time in each |
| volume | of the reactors |
| Cleaning/standby time | no |
| Time without feeding | 6 hours |
| and/or removing | |
| Average hydrolysis time | 60 hours |
| for the whole concept of | |
| four parallel reactors | |

In this example cellulose-based material was fed into the one and the same reactor from which hydrolysed material was simultaneously being removed. One reactor at a time was handled in this manner. No hydrolysed material was removed from nor cellulose-based material was fed into the rest of the reactors.

From the above results one may calculate the following increase in the average enzymatic hydrolysis time of example 1a compared to comparative example 1:

$$( \frac{60 hours}{42 hours} ) \times 100 \% - 100 \% = 43 \%$$

In addition no standby time for cleaning of any of the reactors was needed in example 1a. From the results one can see that the average hydrolysis time can be increased for the same total reactor capacity or total reactor volume when compared to the comparative example.

### Example 1b—Producing Hydrolysed Material

In this example hydrolysed material was produced by enzymatic hydrolysis of cellulose-based material in a manner as disclosed in FIG. 1c. The following parameters were used:

TABLE 3

| | |
|---|---|
| Number of parallel reactors | 4 |
| Volume of each of the reactors | 1000 m³ |
| Feeding/removing time | 5 hours |
| Flow rate | 60 m³/h |
| Filling level of the reactor | lowest level of 60% in |
| volume | the reactor from which |
| | hydrolysed material was |
| | being removed |
| | 90% in the rest of the |
| | reactors |
| Cleaning/standby time | no |
| Time without feeding | 10 hours |
| and/or removing | |
| Average hydrolysis time | 55 hours |
| for the whole concept of | |
| four parallel reactors | |

In this example only two of the reactors in the line of parallel reactors were filled and/or emptied at the time. Cellulose-based material was fed into the one reactor while hydrolysed material was simultaneously being removed from the following reactor in the line of parallel reactors. No hydrolysed material was removed from nor cellulose-based material was fed into the rest of the reactors.

From the above results one may calculate the following increase in the average enzymatic hydrolysis time of example 1b compared to comparative example 1:

$$( \frac{55 hours}{42 hours} ) \times 100 \% - 100 \% = 31 \%$$

In addition no standby time for cleaning of any of the reactors was needed in example 1b. From the results one can see that the average hydrolysis time can be increased for the same total reactor capacity or total reactor volume when compared to the comparative example.

### Comparative Example 2—Producing Hydrolysed Material

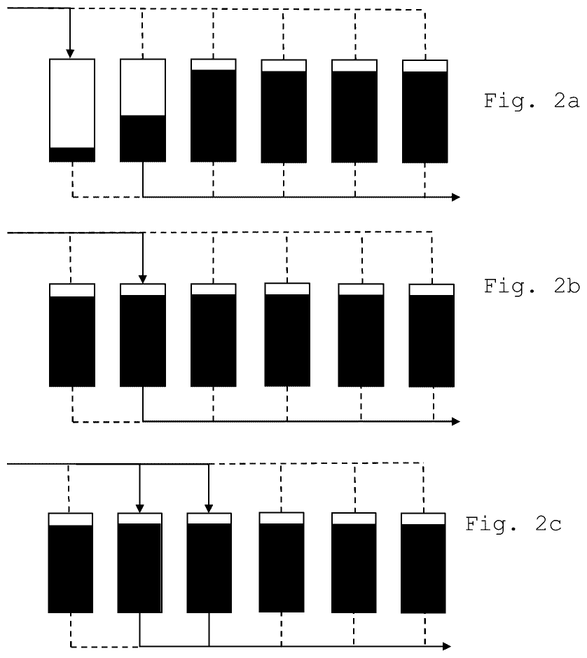

In this comparative example hydrolysed material was produced by enzymatic hydrolysis of cellulose-based material in a manner as disclosed in FIG. 2a. The following parameters were used:

TABLE 4

| | |
|---|---|
| Number of parallel reactors | 6 |
| Volume of each of the reactors | 500 m³ |
| Time interval B | 10 hours |
| (feeding time) | |
| Time interval A | 10 hours |
| (removing time) | |
| Flow rate | 45 m³/h |
| Filling level of the reactor volume | 90%* |
| Cleaning/standby time | 3 hours |
| Time between feeding and | 37 hours |
| removing | |
| Average hydrolysis time | 47 hours |
| for the whole concept of six | |
| parallel reactors | |

*In those reactors where enzymatic hydrolysis was ongoing, and no hydrolysed material was removed nor cellulose-based material was fed

In this comparative example cellulose-based material was fed into the reactors with continuous feeding and emptying of the reactors one by one. One reactor at a time was emptied, cleaned, and the re-filled. I.e. a reactor was completely emptied from hydrolysed material and after the cleaning of the reactor, it was re-filled or fed with cellulose-based material. The reactors were filled up to a value of 90% of the reactor volume.

### Example 2a—Producing Hydrolysed Material

In this example hydrolysed material was produced by enzymatic hydrolysis of cellulose-based material in a manner as disclosed in FIG. 2b. The following parameters were used:

TABLE 5

| | |
|---|---|
| Number of parallel reactors | 6 |
| Volume of each of the reactors | 500 m³ |
| Feeding/removing time | 2 hours |
| Flow rate | 60 m³/h |
| Filling level of the reactor | 90% all the time in each |
| volume | of the reactors |
| Cleaning/standby time | no |
| Time without feeding | 10 hours |
| and/or removing | |
| Average hydrolysis time | 60 hours |
| for the whole concept of | |
| six parallel reactors | |

In this example cellulose-based material was fed into the one and the same reactor from which hydrolysed material was simultaneously being removed. One reactor at a time was handled in this manner. No hydrolysed material was removed from nor cellulose-based material was fed into the rest of the reactors.

From the above results one may calculate the following increase in the average enzymatic hydrolysis time of example 2a compared to comparative example 2:

$$( \frac{60 hours}{47 hours} ) \times 100 \% - 100 \% = 28 \%$$

In addition no standby time for cleaning of any of the reactors was needed in example 2a. From the results one can see that the average hydrolysis time can be increased for the same total reactor capacity or total reactor volume when compared to the comparative example.

### Example 2b—Producing Hydrolysed Material

In this example hydrolysed material was produced by enzymatic hydrolysis of cellulose-based material in a manner as disclosed in FIG. 2c. The following parameters were used:

TABLE 6

| | |
|---|---|
| Number of parallel reactors | 6 |
| Volume of each of the reactors | 500 m³ |
| Feeding/removing time | 2 hours |
| Flow rate | 22.5 m³/h per reactor |
| | (45 m³/h together) |
| Filling level of the reactor | 90% all the time in each |
| volume | of the reactors |
| Cleaning/standby time | no |
| Time without feeding | 4 hours |
| and/or removing | |
| Average hydrolysis time | 60 hours |
| for the whole concept of | |
| six parallel reactors | |

In this example cellulose-based material was fed into the one and the same reactor from which hydrolysed material was simultaneously being removed. Two reactors at a time were handled, i.e. hydrolysed material was removed from two reactors while at the same time being fed with cellulose-based material. No hydrolysed material was removed from nor cellulose-based material was fed into the rest of the reactors.

From the above results one may calculate the following increase in the average enzymatic hydrolysis time of example 2b compared to comparative example 2:

$$( \frac{60 hours}{47 hours} ) \times 100 \% - 100 \% = 28 \%$$

In addition no standby time for cleaning of any of the reactors was needed in example 2b. From the results one can see that the average hydrolysis time can be increased for the same total reactor capacity or total reactor volume when compared to the comparative example.

### Comparative Example 3—Producing Hydrolysed Material

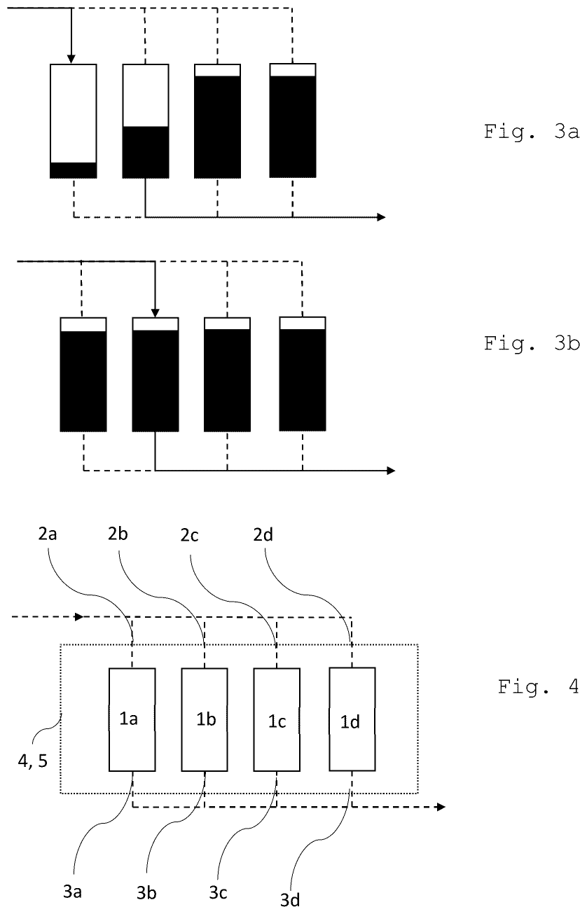

In this comparative example hydrolysed material was produced by enzymatic hydrolysis of cellulose-based material in a manner as disclosed in FIG. 3a. The following parameters were used:

TABLE 7

| | |
|---|---|
| Number of parallel reactors | 4 |
| Volume of each of the reactors | 330 m³ |
| Time interval B | 5 hours |
| (feeding time) | |
| Time interval A | 5 hours |
| (removing time) | |
| Flow rate | 60 m³/h |
| Filling level of the reactor volume | 90%* |
| Cleaning/standby time | 3 hours |
| Time between feeding and | 7 hours |
| removing | |
| Average hydrolysis time | 12 hours |
| for the whole concept of four | |
| parallel reactors | |

*In those reactors where enzymatic hydrolysis was ongoing, and no hydrolysed material was removed nor cellulose-based material was fed

In this comparative example cellulose-based material was fed into the reactors with continuous feeding and emptying of the reactors one by one. One reactor at a time was emptied, cleaned, and the re-filled. I.e. a reactor was completely emptied from hydrolysed material and after the cleaning of the reactor, it was re-filled or fed with cellulose-based material. The reactors were filled up to a value of 90% of the reactor volume.

### Example 3—Producing Hydrolysed Material

In this example hydrolysed material was produced by enzymatic hydrolysis of cellulose-based material in a manner as disclosed in FIG. 3b. The following parameters were used:

TABLE 8

| | |
|---|---|
| Number of parallel reactors | 4 |
| Volume of each of the reactors | 330 m³ |
| Feeding/removing time | 1 hours |
| Flow rate | 60 m³/h |
| Filling level of the reactor | 90% all the time in each |
| volume | of the reactors |
| Cleaning/standby time | no |
| Time without feeding | 3 hours |
| and/or removing | |
| Average hydrolysis time | 20 hours |
| for the whole concept of four | |
| parallel reactors | |

In this example cellulose-based material was fed into the one and the same reactor from which hydrolysed material was simultaneously being removed. One reactor at a time was handled in this manner. No hydrolysed material was removed from nor cellulose-based material was fed into the rest of the reactors.

From the above results one may calculate the following increase in the average enzymatic hydrolysis time of example 3 compared to comparative example 3:

$$( \frac{20 hours}{12 hours} ) \times 100 \% - 100 \% = 67 \%$$

In addition no standby time for cleaning of any of the reactors was needed in example 3. From the results one can see that the average hydrolysis time can be increased for the same total reactor capacity or total reactor volume when compared to the comparative example.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A process or an arrangement, as disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A process for conducting enzymatic hydrolysis of a cellulose-based material to produce hydrolysed material, wherein the process comprises:
providing at least two parallel reactors;
subjecting cellulose-based material to enzymatic hydrolysis in each of the at least two reactors;
removing hydrolysed material from at least one reactor during a time interval A;
feeding cellulose-based material into at least one reactor during a time interval B;
controlling that in none of the at least two reactors the filling level of the reactor is below a value of 40% of the reactor volume; and
wherein during the process there is a time period for each of the reactors when no cellulose-based material is fed into the reactor or hydrolysed material is removed from the reactor at the same time.

2. The process of claim 1, wherein the average hydrolysis time in each of the reactors is 6-90 hours.

3. The process of claim 1, wherein the hydrolysed material is removed in an amount such that the filling level of the reactor remains at a value of at least 40% of the reactor volume.

4. The process of claim 1, wherein the process comprises constantly keeping the filling level in each of the reactors at a value of 40-99% of the reactor volume.

5. The process of claim 1, wherein the process comprises constantly keeping the filling level in each of the reactors at a value of 85-99% of the reactor volume.

6. The process of claim 1, wherein the reactors are batch-type reactors.

7. The process of claim 1, wherein the average flow rate for feeding cellulose-based material into a reactor and for removing hydrolysed material from a reactor is essentially equal.

8. The process of claim 1, wherein removing hydrolysed material from at least one reactor and feeding cellulose-based material into at least one reactor are carried out such that there is at least one reactor at a time from which no hydrolysed material is removed from and/or to which no cellulose-based material is fed into.

9. The process of claim 1, wherein the process comprises providing at least three parallel reactors and removing hydrolysed material simultaneously from more than one reactor during the time interval A.

10. The process of claim 1, wherein the process comprises providing at least three parallel reactors and feeding cellulose-based material simultaneously into more than one reactor during the time interval B.

11. The process of claim 1, wherein removing hydrolysed material from a reactor and feeding cellulose-based material into a reactor are carried out simultaneously in one and the same reactor.

12. The process of claim 1, wherein hydrolysed material is removed from at least one reactor while simultaneously cellulose-based material is fed into at least one reactor, which is different from the reactor from which hydrolysed material is being removed.

13. The process of claim 1, wherein the total consistency in each of the reactors is essentially constant throughout the process.

14. The process of claim 1, wherein the process comprises providing 2-20 parallel reactors.

15. An arrangement for conducting enzymatic hydrolysis of a cellulose-based material to produce hydrolysed material, wherein the arrangement comprises:
at least two parallel reactors, wherein each of the reactors comprises an outlet configured to remove hydrolysed material from the reactor and an inlet configured to feed cellulose-based material into the reactor; and wherein each of the at least two reactors is configured to subject cellulose-based material to enzymatic hydrolysis;
a measurement system configured to produce measurement information indicative of the filling level in each of the reactors; and
a control system configured to control that in none of the at least two reactors the filling level of the reactor is below a value of 40% of the reactor volume, and to control that each of the reactors has a time period during which no cellulose-based material is fed into the reactor or hydrolysed material is removed from the reactor at the same time.
